(19) United States
(12) Plant Patent Application Publication
(10) Pub. No.: US 2013/0025013 P1
(43) Pub. Date: Jan. 24, 2013

(54) Guzmania hybrid named 'JUNO'

(50) ;

(71) Applicants: Elly Bak, Rijsenhout (NL); Nicolaas D.M. Steur, Oude Niedorp (NL)

(72) Inventors: Elly Bak, Rijsenhout (NL); Nicolaas D.M. Steur, Oude Niedorp (NL)

(73) Assignee: Corn.Bak B.V.

(21) Appl. No.: 13/137,067

(22) Filed: Jul. 19, 2011

(51) Int. Cl. A01H 5/00

(52) U.S. Cl. PLT/371

(57) ABSTRACT

A new and distinct Guzmania plant named ‘JUNO’ characterized by solid growth habit; funnel-form rosette plant, measuring about 57 cm in height (above the pot when flowering); numerous, green color foliage, measuring about 45 cm in length and about 3.0 cm to 3.5 cm in width; superior floral bract production; bracts have a unique pinkish-red inflorescence (RHS 47A); compound inflorescence, measuring from about 17 cm in height and about 18 cm in diameter; and long-lasting habit.

### LATIN NAME OF THE GENUS AND SPECIES OF THE CLAIMED PLANT

Guzmania sp.

### VARIETY DENOMINATION

‘JUNO’

### BACKGROUND OF THE INVENTION

The present invention relates to a new and distinct hybrid cultivar of Guzmania plant, botanically known as Guzmania sp., of the family Bromeliaceae, and hereinafter referred to as ‘JUNO’.

Guzmania is native to tropical America. Guzmania is predominantly epiphytic with a few terrestrial species and is native to the tropics. For the most part, species vary in diameter from 7 or 8 inches to 3 or 4 feet and have rosettes of glossy, smooth-edged leaves. Floral bracts of Guzmania frequently have brilliant colors and may last for many months. The range of colors for Guzmania is generally from yellow through orange but may also include flame red and red-purple. White or yellow, tubular, three-petalled flowers may also appear on a stem or within the leaf rosette but are usually short-lived. Guzmania may be advantageously grown as pot plants for greenhouse or home use. Typically, the plants are shaded from direct sunlight. During the spring to autumn period, the central vase-like part of the leaf rosette is normally filled with water.

The new Guzmania ‘JUNO’ was discovered and selected by the inventors in 2002 as a single flowering plant in a controlled breeding program in Assendelft, The Netherlands. The new Guzmania ‘JUNO’ originated from a naturally occurring mutation of Guzmania (hybrid) ‘LUNA’ (unpatented).

Asexual reproduction of the new Guzmania cultivar was first performed by off-shoots beginning in 2003 and then by tissue culture beginning in 2005 in Assendelft, The Netherlands, with first flowering after asexual reproduction occurring in 2009 in Assendelft, The Netherlands. Asexual reproduction of the Guzmania ‘JUNO’ has demonstrated that the combination of characteristics as herein disclosed for the new cultivar are firmly fixed arid retained through successive generations of asexual reproduction. The new cultivar reproduces true to type.

Methods for cultivation and crossing of Guzmania are well known. For a detailed discussion, reference is made to the following publications, which are incorporated herein by reference: Benzing, David H., THE BIOLOGY OF THE BROMELIADS, Mad River Press, Inc., Eureka (1980); Zimmer, Karl, BROMELIEN, Verlag, Paul Parey, Berlin (1986); and Rauh, Werner, BROMELIEN, Verlag Eugen Ulmer, Stuttgart (1981).

### BRIEF SUMMARY OF THE INVENTION

The following traits have been repeatedly observed and are determined to be unique characteristics of ‘JUNO’ which in combination distinguish this Guzmania as a new and distinct cultivar:

1. Solid growth habit;

2. Funnel-form rosette plant, measuring about 57 cm in height (above the pot when flowering);

3. Numerous, green color foliage, measuring about 45 cm in length and about 3.0 cm to 3.5 cm in width;

4. Superior floral bract production;

5. Bracts have a unique pinkish red inflorescence (RHS 47A);

6. Compound inflorescence, measuring from about 17 cm in height and about 18 cm in diameter; and

7. Long-lasting habit.

Of the many commercial cultivars known to the present inventors, the most similar in comparison to the new Guzmania ‘JUNO’ is the Guzmania hybrid ‘FREYA’ (patented, U.S. Plant Pat. No. 21,461). Plants of the new Guzmania ‘JUNO’ differ from plants of ‘FREYA’ primarily in the following characteristics:

Plants of ‘JUNO’ produce compound inflorescence which are pinkish red (closest to RHS 47A) in color, whereas plants of ‘FREYA’ produce compound inflorescence which are red-purple (closest to RHS 61B) in color.

### BRIEF DESCRIPTION OF THE PHOTOGRAPHS

The accompanying photographs illustrate the overall appearance of the new Guzmania ‘JUNO’ showing the colors as true as is reasonably possible with colored reproductions or this type. Colors in the photographs may differ slightly from the color values cited in the detailed botanical description which accurately describes the color of ‘JUNO’.

FIG. 1 shows a side view perspective of the primary and top bracts produced by a typical potted, flowering plant of ‘JUNO’, at 12 months of age from potting size.

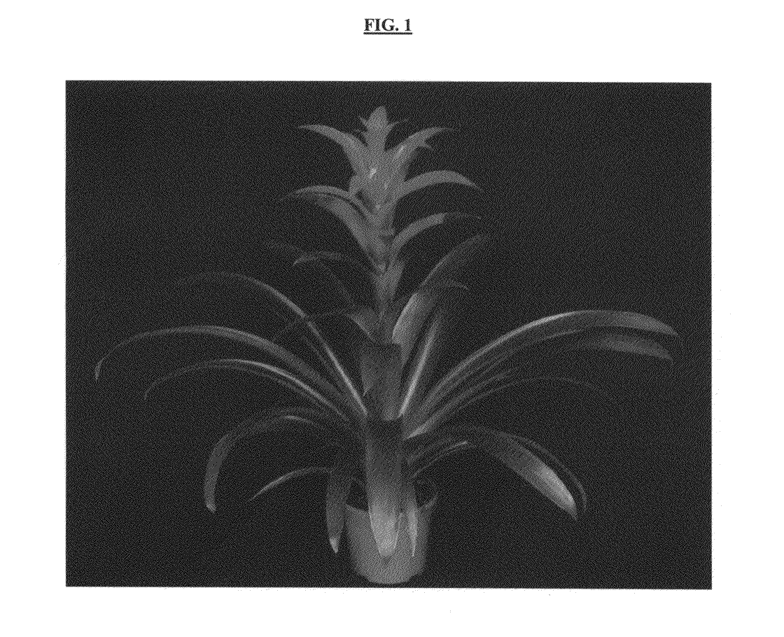

FIG. 2 shows a close-up top view perspective of the inflorescence and top bracts produced by a typical potted, flowering plant of ‘JUNO’, at 12 months of age from potting size.

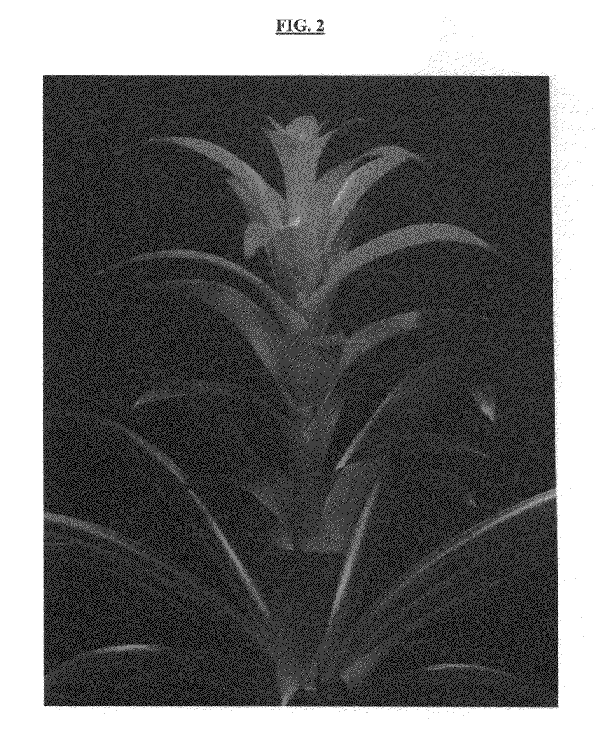

### DETAILED BOTANICAL DESCRIPTION

‘JUNO’ has not been tested and observed under all possible environmental conditions. The phenotype of the new cultivar may vary with variations in environment such as temperature, light intensity, frequency of fertilization, composition of fertilizer, flowering treatment, day length and humidity, without any change in the genotype of the plant.

For example, substantial differences in plant height and diameter, and number of leaves can result depending on the size of the plant at the time that flowering is induced. Since treatment to induce flowering disrupts normal watering and fertilization regimens, flowering treatment of relatively smaller plants adversely affects the growth of the plant.

The afore mentioned photographs, together with the following observations, measurements and values describe the new Guzmania ‘JUNO’ as grown in a greenhouse in Assendelft, The Netherlands, under conditions which closely approximate those generally used in commercial practice. Plants of ‘JUNO’ were grown in a greenhouse with day temperatures ranging from 20° C. to 28° C. and night temperatures ranging from 18° C. to 23° C. No artificial lighting or photoperiodic treatments were conducted, but plants of ‘JUNO’ are forced into flowering. The following fertilizer is added when growing plants of ‘FREYA’: 1 part nitrogen, 0.6 parts phosphor, 2 parts Kalium and 0.1 parts magnesium.

Color references are made to The Royal Horticultural Society Colour Chart (R.H.S.), 2001 edition, except where general colors of ordinary significance are used. Color values were taken under daylight conditions in a greenhouse in Assendelft, The Netherlands. The age of the plants of ‘JUNO’ described is about 17 weeks after flowering treatment.
Classification:
   Botanical.—Guzmania sp.
Parentage: naturally occurring mutation of Guzmania (hybrid) ‘LUNA’ (unpatented)
Plant: General Appearance and Form: Height: About 57 cm (when flowering) Width: About 70 cm Shape: Funnel form rosette Growth habit: Stemless Plant Vigor: Good Flowering Season: A fully grown plant can flower year round, starting 17 weeks after induction of natural light or through flowering treatment. Cold Tolerance: Frost tender. Temperatures below 5° C. may damage plants. Fragrance: None.
Foliage: Quantity: About 18 (depending on the size or the plant) Size of Leaf: Length: About 45 cm (when flowering) Width: About 3.0 to 3.5 cm Overall Shape: Linear-lanceolate Apex Shape: Acuminate Base Shape: Strap-like around central axis Margin: Entire Texture (both surfaces): Smooth Orientation: Leaf blades arch continuously from base. Color: Leaf color can vary somewhat depending on growing conditions Mature: Upper surface: Green, RHS 147A Under surface: Green, RHS 137A Immature: Upper surface: Green, RHS 147A Under surface: Green, RHS 137A Venation: None.
Inflorescence: Borne: Erect stalks Shape: Compound Size: Length (increases with maturity): About 17 Diameter: About 18 cm Time of Bloom: A fully grown plant can produce an inflorescence containing about 100 flowers (depending on the size of the plants), and can bloom the whole year starting about 17 weeks after natural induction or through flowering treatment. Duration of Bloom: Each flower blooms one (1) day and the total blooming of the whole inflorescence is about five (5) weeks. Petals: Number: 3 per flower Length: About 6.0 cm Width: About 0.5 cm Overall Shape: Ligulate Apex Shape: Obtuse Base Shape: Fused Color: Upper and Under surfaces: Yellow, RHS 8C Sepals: Number: 3 per flower Length: About 3.5 cm Width: About 0.5 cm Overall Shape:Ligulate Apex Shape: Acute Base Shape: Fused Color: Translucent.
Bracts: Scape Bracts: Quantity: About 12 Arrangement: Alternate Size: Length: About 27 cm(lowest) to about 18 cm (scape bracts positioned just below the primary bracts). Width: About 3.5 cm Overall shape: Lanceolate Apex shape: Acute Base shape: Fused Margin: Entire Texture: Smooth Color: Upper surface: Green, RHS 147A, with red-purple, RHS 61B. Scape bracts become more red-purple in color, closer to the primary bracts. Under surface: Green, RHS 137A, with red-purple, RHS 61B. Scape bracts become more red-purple in color, closer to the primary bracts. Primary Bracts: Quantity: About 9 Arrangement: Alternate Size: Length: About 17 cm (lowest) to about 9 cm (primary bracts become shorter closer to the top of plant) Width: About 3.0 cm Overall shape: Lanceolate Apex shape: Acute Base shape: Fused Margin: Entire Texture: Smooth Color: Upper and Under surfaces: pinkish-red, RHS 47A Floral bracts: Disposed within the inflorescence.
Productive organs:
   Androecitan.—Stamen: Number: 6 per flower Length: About 5.0 cm Diameter: About 1.0 mm Color: White, (too small to qualify RHS value) Anther: Length: About 0.6 cm Color: Cream, (too small to qualify RHS value) Pollen: Amount: Scarce Color: Cream (too small to qualify RHS value).
   Gynoecium.—Pistil: Number: 1 per flower Length: About 5.5 cm Stigma: Shape: 3-parted Width: About 4.0 mm Color: White, (too small to qualify RHS value) Style: Length: About 4.5 cm Color: White, (too small to qualify RHS value) Ovary: Position: Superior Shape: Conical Length: About 0.6 cm Diameter: About 0.3 cm Color: Light green, RHS 144C.
Seeds/fruit: Sterile hybrid, therefore, no seed or fruit produced
Disease/pest resistance: No observations made.
Disease/pest susceptibility: No observations made.

We claim:

1. A new and distinct Guzmania plant named ‘JUNO’, substantially as illustrated and described herein.
